(19) United States
(12) Patent Application Publication
(10) Pub. No.: US 2018/0317789 A1
(43) Pub. Date: Nov. 8, 2018

(54) Devices and Methods for Remote Monitoring of Heart Activity

(71) Applicant: Signature Medical, Inc., Boston, MA (US)

(72) Inventors: Terrance J. Ransbury, Chapel Hill, NC (US); Kenneth C. Armstrong, Cary, NC (US); Omar Amirana, Cambridge, MA (US)

(21) Appl. No.: 15/969,213

(22) Filed: May 2, 2018

Related U.S. Application Data
(60) Provisional application No. 62/500,003, filed on May 2, 2017.

(51) Int. Cl. A61B 5/024; A61B 5/0245; A61B 5/0456; A61B 5/00; A61B 7/04

(52) U.S. Cl. CPC A61B 5/02438; A61B 5/0245; A61B 5/0456; A61B 7/04; A61B 5/7203; A61B 5/0022; A61B 5/02405

(57) ABSTRACT

Devices and methods for remote monitoring of heart activity are disclosed. In some embodiments, a wearable heart monitoring devices for monitoring heart activity comprises one or more acoustic sensors, including at least one microphone configured to operate in a frequency range related to human hearing, and at least one accelerometer configured to operate in the range of low or sub-audible frequencies.

### RELATED APPLICATIONS

The present application claims priority to and the benefit of U.S. Provisional Application No. 62/500,003, filed May 2, 2017, which is incorporated herein by reference in its entirety.

### FIELD

The present disclosure relates to methods and systems for remote monitoring of heart activity.

### BACKGROUND

Obtaining and monitoring of acoustic signals to diagnose cardiac function is as old as the stethoscope. Many cardiac abnormalities have been identified using the cardiac acoustic signature including valve anomalies and heart failure. Traditionally, a physician uses either a mechanical or an electronic stethoscope, in contact with the patient, to acquire the acoustic signal. The physician makes a diagnosis based upon training and experience in real time. Also traditional is that this examination typically takes place while the patient is at the care facility (e.g. physician's office, clinic, or hospital). However, the traditional methods suffer from multiple issues. Accordingly, there is still a need for simple and effective systems and methods for heart monitoring that can be used remotely, if necessary.

### SUMMARY

In some aspects, the present disclosure provides a wearable heart monitoring device for monitoring heart activity comprising one or more acoustic sensors, including at least one microphone configured to operate in a frequency range related to human hearing, and at least one accelerometer configured to operate in the range of low or sub-audible frequencies.

In some embodiments, the wearable heart monitoring device further comprises a processor in communication with the one or more acoustic sensors, the processor being configured to analyze data collected by the one or more sensors to determine a condition of a heart. In some embodiments, analyzing the collected data includes noise reduction and location of clinically standard heart sounds using peak detection and frequency analysis. In some embodiments, the processor is positioned within the wearable heart monitoring device. In some embodiments, the processor is remote form the heart monitoring device and is configured to communicate with the one or more acoustic sensors using a wireless connection. In some embodiments, the processor is configured to sample data from the one or more acoustic sensors at a rate of 12 to 24-bits per sample. In some embodiments the wearable heart monitoring device further comprises one or more data acquisition components configured to supplement the acoustic data collected by the one or more acoustic sensors with additional data relating to the heart. In some embodiments, one of the one or more data acquisition components is one or more EKG electrodes. In some embodiments, the one or more EKG electrodes are configured to collect EKG vectors to produce at least one EKG signal that can be analyzed for heart rate, R-wave detection, heart-rate variability, or the presence of arrhythmia. In some embodiments, one of the one or more data acquisition components is at least one gyroscope configured to determine an orientation of the heart monitoring device in space.

In some embodiments, the gyroscope is configured to account for changes in blood flow through the heart relating to gravity as a body moves through space. In some embodiments, the gyroscope utilizes as least three axes to determine orientation of the heart monitoring device. In some embodiments, the wearable heart monitoring device further comprises a sensor recharger configured to refresh the acoustic sensor using wired or wireless communication. In some embodiments the at least one microphone is configured to operate in the range between about 4 KHz and about 1 KHz. In some embodiments the at least one accelerometer is configured to operate in the range between about 1 KHz and about 0.5 Hz.

In some aspects, the present disclosure provides a wearable heart monitoring device for monitoring heart activity comprising one or more acoustic sensors, including at least one microphone configured to operate in a frequency range related to human hearing, at least one accelerometer configured to operate in the range of low or sub-audible frequencies, and at least one gyroscope configured to determine an orientation of the heart monitoring device in space, wherein data collected by the one or more acoustic sensors and the at least one gyroscope is analyzed to determine a condition of a heart of a patient wearing the heart monitoring device.

In some embodiments, the wearable heart monitoring device further comprises one or more data acquisition components configured to supplement the acoustic data collected by the one or more acoustic sensors with additional data relating to the heart. In some embodiments, one of the one or more data acquisition components is one or more EKG electrodes.

In some aspects, the present disclosure provides a method for monitoring heart activity comprising collecting acoustic data using one or more acoustic sensors, the one or more acoustic sensors including at least one microphone configured to operate in a frequency range related to human hearing and at least one accelerometer configured to operate in the range of low or sub-audible frequencies, and analyzing data collected by the one or more sensors to determine a condition of a heart using a processor. The method for determining the condition of a heart of claim 19, wherein analyzing the collected data includes noise reduction and location of clinically standard heart sounds using peak detection and frequency analysis.

### BRIEF DESCRIPTION OF THE DRAWINGS

The presently disclosed embodiments will be further explained with reference to the attached drawings, wherein like structures are referred to by like numerals throughout the several views. The drawings shown are not necessarily to scale, with emphasis instead generally being placed upon illustrating the principles of the presently disclosed embodiments.

FIG. 1 is a high-level block diagram of an embodiment of the system;

FIG. 2 is a high-level block diagram of an embodiment of the system where the sensor communicates via telemetry to a dedicated heart failure workstation located in the hospital or clinic;

FIG. 3 is a block diagram of an embodiment of a wearable sensor and charger;

FIG. 4 is a block diagram of an embodiment of the system where the means to communicate to and from the cloud are located in the sensor recharger;

FIG. 5 is an exemplary block diagram showing the software components of the system residing in the cloud;

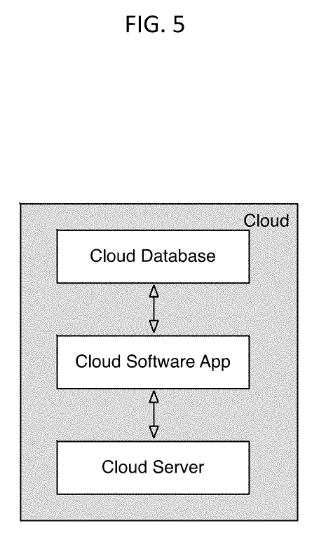

FIG. 6 is a plan view showing an embodiment of the wearable acoustic sensor;

FIG. 7 is an example of a recording of normal heart activity;

FIG. 8 is an example of a recording of a patient in congestive heart failure. Note the significant and prominent S3 heart sound typical of a heart failure patient;

FIG. 9 is an embodiment of a heart monitoring device; and

FIG. 10 is a diagram showing an exemplary computer system suitable for use with the methods and systems of the present disclosure.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

The following description provides exemplary embodiments only, and is not intended to limit the scope, applicability, or configuration of the disclosure. Rather, the following description of the exemplary embodiments will provide those skilled in the art with an enabling description for implementing one or more exemplary embodiments. It will be understood that various changes may be made in the function and arrangement of elements without departing from the spirit and scope of the presently disclosed embodiments

Specific details are given in the following description to provide a thorough understanding of the embodiments. However, it will be understood by one of ordinary skill in the art that the embodiments may be practiced without these specific details. For example, systems, processes, and other elements in the presently disclosed embodiments may be shown as components in block diagram form in order not to obscure the embodiments in unnecessary detail. In other instances, well-known processes, structures, and techniques may be shown without unnecessary detail in order to avoid obscuring the embodiments.

Also, it is noted that individual embodiments may be described as a process which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process may be terminated when its operations are completed, but could have additional steps not discussed or included in a figure. Furthermore, not all operations in any particularly described process may occur in all embodiments. A process may correspond to a method, a function, a procedure, a subroutine, a subprogram, etc. When a process corresponds to a function, its termination corresponds to a return of the function to the calling function or the main function.

Subject matter will now be described more fully with reference to the accompanying drawings, which form a part hereof, and which show, by way of illustration, specific example aspects and embodiments of the present disclosure. Subject matter may, however, be embodied in a variety of different forms and, therefore, covered or claimed subject matter is intended to be construed as not being limited to any example embodiments set forth herein; example embodiments are provided merely to be illustrative. The following detailed description is, therefore, not intended to be taken in a limiting sense.

The present disclosure relates to medical procedures involving heart failure. In particular, methods and systems are provided for remote monitoring of a heart of a patient and the ability to deliver personalized and precise care. Acoustic information is gathered and analyzed in order to provide a patient and/or health care provider and physician notifications with the intent of reducing costly hospital admissions. In some embodiments, a system and method are provided for obtaining acoustic cardiac information from a patient, as shown and described herein. The system can include means for one or more wearable acoustic transducers, means for acquiring acoustic signals from the patient, means for transmitting the acquired acoustic signals for analysis, means for notifying the patient of cardiac status, and means for notifying the physician of the patient's cardiac status. Active and directional noise cancellation can be used to remove ambient noise.

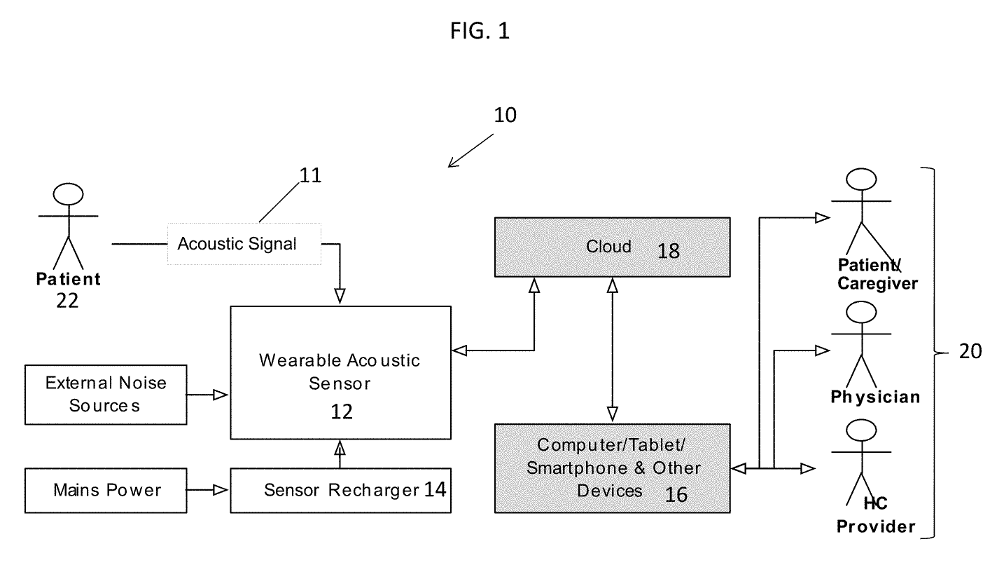

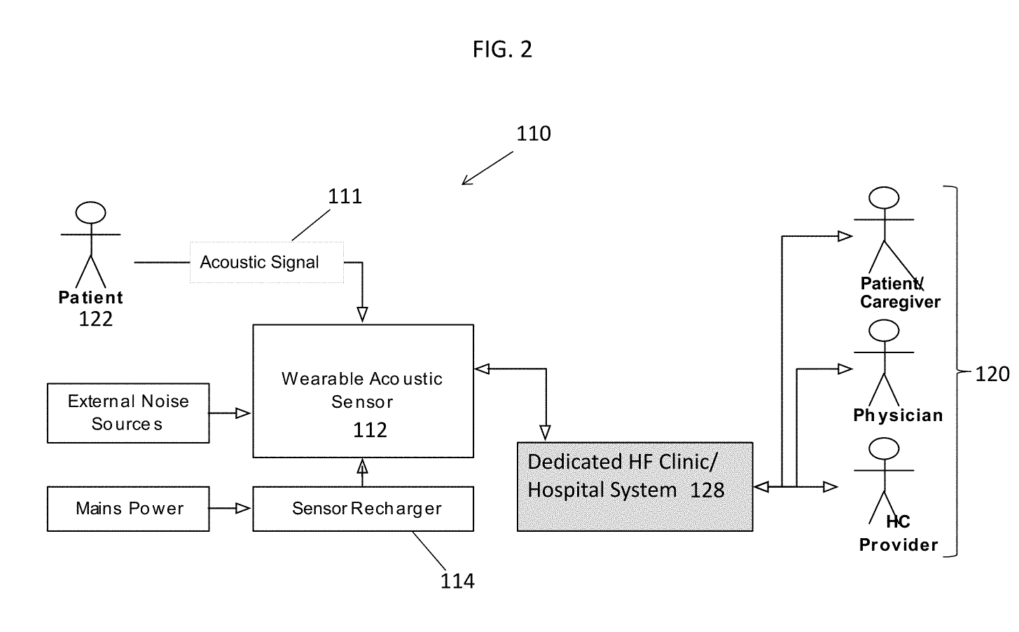

In some embodiments, a wearable heart monitoring device 10, 110 is shown for detecting acoustic signal 11, 111 related to heart activity of a patient 22, 122. As shown in FIG. 1, the heart monitoring device 10 can include one or more wearable acoustic sensors 12, a sensor recharger 14, cloud-based analysis software, and a communications device 16, such as a computer, a smartphone, a tablet, or other device. As shown in FIG. 2, the heart monitoring device 110 can include one or more wearable acoustic sensors 112, and a sensor recharger 114. The heart monitoring device can communicate using a variety of modalities, including through a network, such as a cloud 18, shown in FIG. 1, and telemetry to a dedicated heart failure workstation 128 located in the hospital or clinic, as shown in FIG. 2. The acoustic and/or other physiological information gathered by the heart monitoring device can be transmitted to a user 20, 120, including the patient, and a doctor or other health care provider. The information can be useful in determining whether or not intervention by the healthcare provider is warranted. Appropriate intervention can prevent re-hospitalization thus saving the healthcare system time and money.

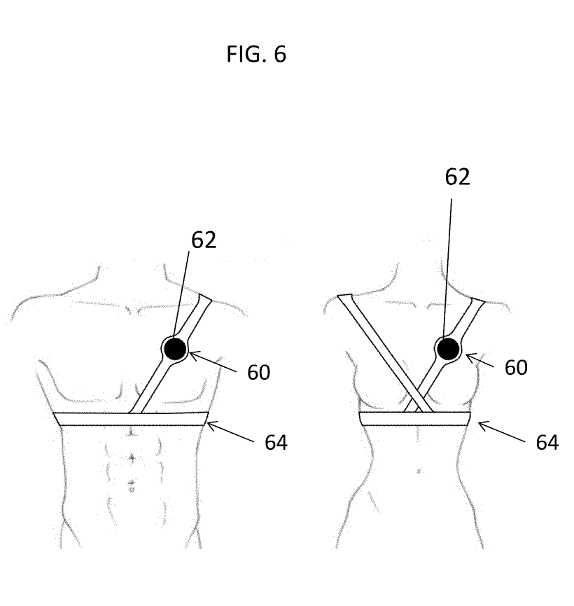

In reference to FIG. 6, the heart monitoring device or acoustic sensor 60 can be housed inside a housing 62. The patient can wear the heart monitoring device either continuously or periodically during the monitoring session, which can last minutes, days or weeks. The heart monitoring device can be held in place by an adhesive or a wearable mechanism 64 as illustrated in FIG. 6. This mechanism can be repeatable and suitable for heart failure patients that may have lost dexterity and cognitive function. The heart monitoring device location attribute of the mechanism can also be adjusted by a physician after mapping for optimal sound amplitude and fidelity.

In some embodiments where the patient holds the heart monitoring device, or where the wearable mechanism is adjustable, the heart monitoring device can analyze the quality of the received signals (with respect to noise) and if non-optimal, the heart monitoring device can give audible, visual signal information, or other feedback information back to the patient or physician to adjust the sensor position. In addition, the heart monitoring device can detect movement of the device and further enhance the optimization of sensor placement.

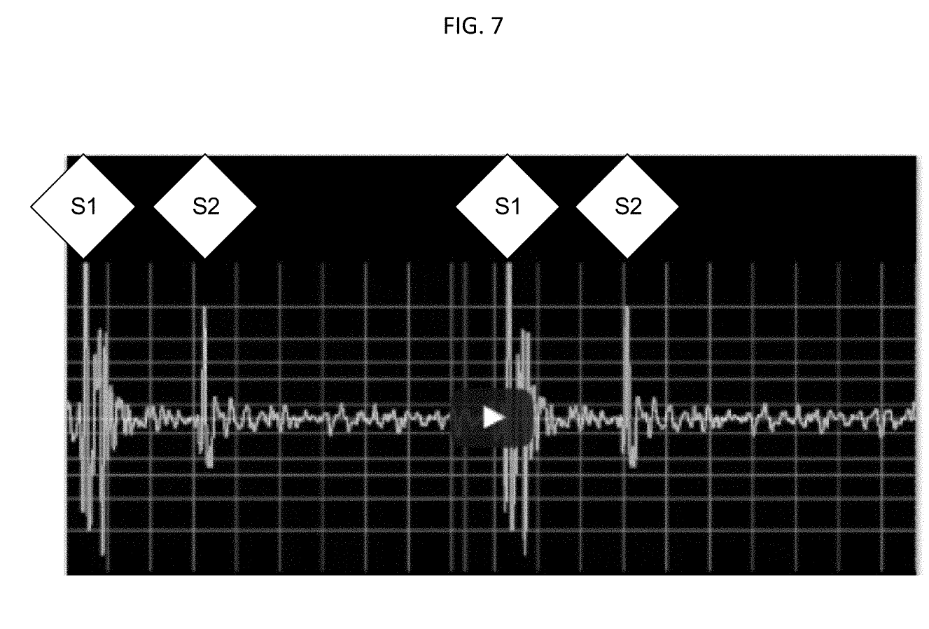

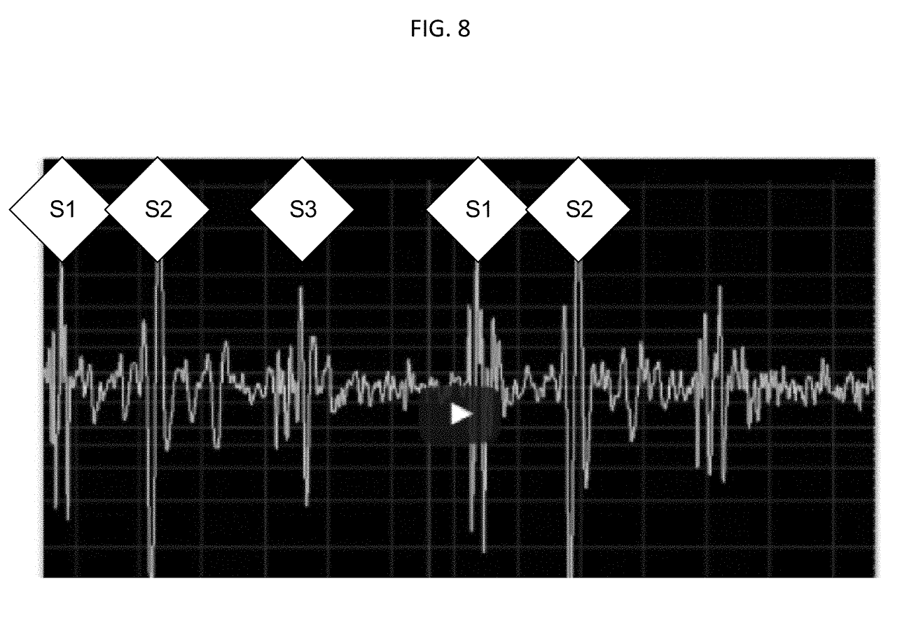

The acoustic sensor can be configured to capture one or more acoustic signals that contain information on cardiac and/or respiratory function, both of which can be used when diagnosing the condition of heart failure patients. Typical acoustic waveforms of normal heart function and heart failure are shown respectively in FIGS. 7 and 8.

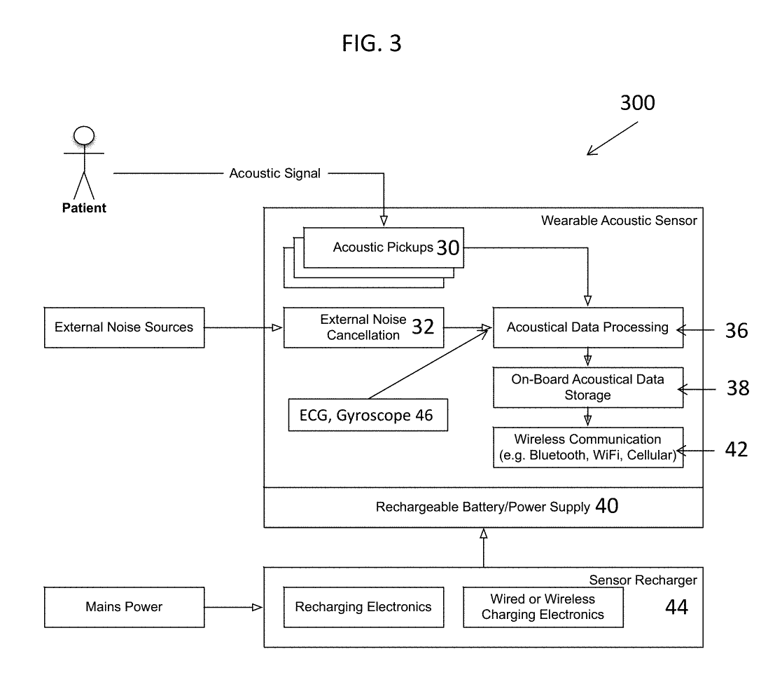

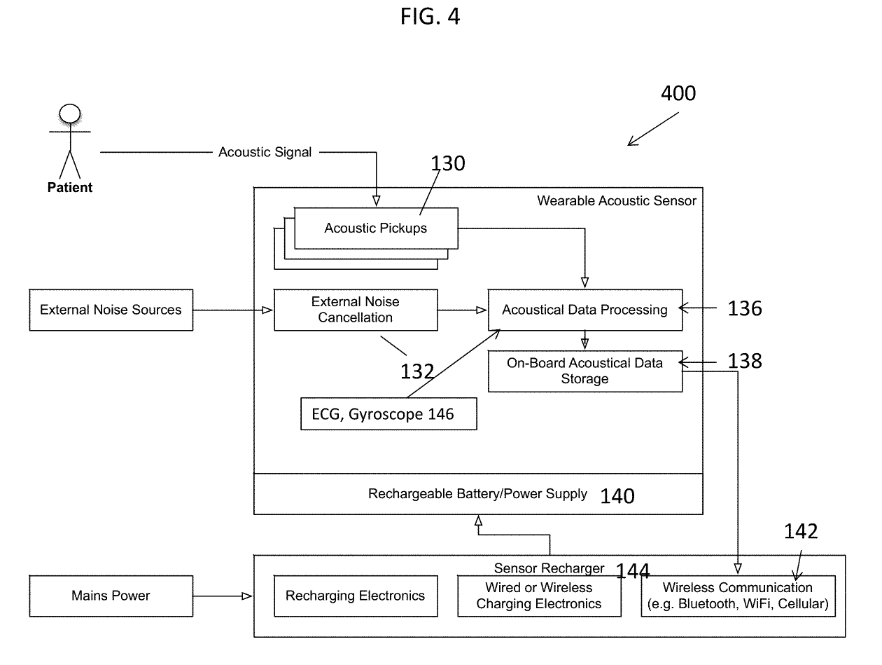

FIGS. 3 and 4 illustrate embodiments of heart monitoring devices used for detecting a heart condition or heart failure. The acoustic sensors 300, 400 can include acoustic pickups 30, 130, such as microphones, accelerometers or both. The microphones are designed for human hearing and as such operate up to 20 KHz but are optimized from 4 KHz down to about 1 KHz. The accelerometers can supplement the acoustic information by capturing low or sub-audible frequencies below 1 KHz down to as low as 0.5 Hz. To best extend the acoustic range of the overall sensor, the accelerometer(s) can be placed either within the microphone or directly adjacent to it and as close to the skin layer as possible.

In some embodiments, multiple microphones and accelerometers can also provide some redundancy in the recordings and lessen the need for precise placement of the sensor on the torso. This can be useful in embodiments where the patient is responsible for placing the sensor herself or himself, knowing this method is prone to variation error.

External noise cancellation components 32, 132 can be optional, but can increase system fidelity and reliability. They can be implemented by one or more microphones pointed away from the torso to maximize sensitivity to ambient noise sources. Their performance specifications should mirror the acoustic pickups for the audible frequencies because ambient noise is in the same range but unwanted and therefore considered noise.

Additional data acquisition components that can be used to supplement the acoustic information include, but are not limited to, EKG electrodes, an EKG amplifier, and a gyroscope 46, 146.

The EKG system can collect one or more standard EKG vectors, such a Lead I, II or III. Standard EKG electrodes with adhesive, such as those available from 3M (Red Dot™) can be used. Depending upon the number of electrodes, a single unifying adhesive can be devised so that the patient can place fewer objects, hopefully maintaining repeatability over time.

The EKG data collected can be diagnostic-quality with frequency endpoints of 0.5 to 100 Hz or less, 10 Hz to 40 Hz, if just R-wave detection is sufficient. The EKG signal can be evaluated and analyzed for heart rate, R-wave detection, heart-rate variability, or the presence of arrhythmia. The R-wave is synchronous to the S1 heart sound and therefor helpful in S1 and S2 identification. One important arrhythmia feature would be the detection of atrial fibrillation, which can be used to inhibit acoustic recordings that would otherwise be meaningless in its presence due to variable chamber filling rates.

In some embodiments, one or more gyroscopes can be used to determine the orientation of the heart monitoring device in space and therefore when attached to the patient, the patient's body position. In some embodiments, a 3-or-more axis gyroscope can be employed. As an inclinometer, the sensor can then add the body position to the recorded data. This is important because gravity changes the heart's position in the torso and this changes the blood flow and this is expected to alter the acoustics.

The combination of the accelerometers and the gyroscope can detect gross and fine motion of the sensor. Detection of motion can be used as a qualifier to recording and a decision can be made in software to delay or inhibit sensor recording in the presence of motion to reduce the amount of data and data analysis. Sensing while in motion is likely noisy and very likely not usable for patient diagnosis. Logically, the absence of motion can be used as a qualifier to start or resume recording after motion has been detected. Both techniques of using motion sensing can be useful in reducing the amount of data to be stored, transferred, and analyzed.

Following amplification of the acoustic and/or other physiological waveforms, digital sampling through an analog-to-digital converter can be performed by circuitry in the sensor prior to storage in on-board memory, for example temporarily, and eventual telemetry for analysis. Sampling rates for the accelerometer can vary. In some embodiments, a sampling rate can range between 12 to 16-bits or 12 to 24-bits per sample for adequate fidelity, at a rate of at least 3 KHz (two times the high frequency range of respiratory signals), and a minimum of 30 seconds of continuous recording at least two times per day for up to 14 days. Plausible ranges for parameters that can influence data storage capacity include but is not limited to frequency ranges from 3 KHz to 44 KHz (audiofile range), recording durations from 10 seconds to 4 or more minutes, recording sessions from 1 to 4 times daily (morning, meals, evenings) to continuous for several days, and periodically from once a week to daily monitoring for the life of the patient.

In some embodiments, the acoustic sensors can include data processing functionality 36, 136, as shown in FIGS. 3 and 4. In some embodiments, the acoustic data may be wirelessly communicated to another device with data processing capabilities.

The acoustic sensors can include on-board data storage device 38, 138. Data storage capacity can be estimated. In some embodiments, sampling at 12 to 16-bits or 12 to 24-bits per sample for adequate fidelity, at a rate of at least 3 KHz (two times the high frequency range of respiratory signals), and a minimum of 30 seconds of continuous recording at least two times per day for up to 14 days would require over 40 Mbits of data storage at a minimum. For example, specifications can include 16-bits or, in some embodiments, 24-bits per sample at a rate of 10 KHz for 120 seconds, four times per day for 14 days, which can require 1 Gbits of data storage. This set of parameters can allow for better signal processing to reduce noise from ambient sounds or sensor movement.

Further acoustical signal processing is optional with respect to being done in the sensor or external to the sensor. In some embodiments, processing can include noise cancellation, either active or passive as described above.

On-board acoustical data storage can be implemented in either volatile (RAM) or non-volatile memory (i.e. flash) according to performance requirements, and the availability of data-retention power (battery backup or mains power). Data retention requirements can be minimal if a communication link to the rest of the system is present or extensive if time between transmission is long (i.e. several days). Additional data storage beyond the 1 Gbits described above would be required for higher sampling rates, more frequent recordings, or recordings of longer duration.

Wireless communications module 42 as shown in FIG. 3 can be included in the wearable acoustic sensor to transfer the recorded data to the remainder of the system for processing and reporting back to the healthcare provider. In some embodiments, a wireless communication module 142 can be external to the acoustic sensor, and can be included, for example, in the sensor charger 144, as shown in FIG. 4

Common forms of wireless communications include short range technologies such as Bluetooth™ and WiFi. Either of these technologies would be suitable to transfer information from the sensor to the base station (for example, sensor recharger, computer, or smart phone) with complementary technology. The data communication range of these technologies could easily be enough for a home-based product. For longer communication ranges, cellular phone technology can be an option that would reduce the need for an intermediary device such as a smartphone or computer. In such an embodiment, the instrument can connect automatically to the cellular network to transmit the recorded data.

In some embodiments, the acoustic sensor can include a rechargeable power supply 40, 140, which can be charged using a sensor charger 44, 144. The sensor charger 44, 144 may include recharging electronics, wired or wireless charging electronics.

As shown in FIGS. 3 and 4, the heart monitoring device needs to adequately deal with external noise sources and minimize their detrimental effect on the recording. In some embodiments, the device can include active noise-cancellation-and-suppression technology. Active noise cancellation can take advantage of knowing that the ambient noise comes directionally from outside of the body hence this noise signal can be recorded and then subtracted from the sensor recording. In some embodiments, the device can include a traditional bandpass filtering.

In some embodiments, the sensor recharger can be used to refresh the acoustic sensor and can be implemented with wired or wireless technology. The sensor recharger is optional and may not be necessary in embodiments where the sensor is low-cost and low-power enough to be disposable.

Due to power constraints, the acoustic sensor can perform little if any signal processing, with less being beneficial to the sensor longevity (see FIGS. 2 and 3). In some embodiments, most of the signal analysis can be done remotely, for example in the cloud, both for power-saving reasons and product maintenance reasons, being that the cloud is much simpler to upgrade.

A determination of usage by the patient population will have a dramatic impact on system design. There are basically two different embodiments possible with the sensor: adhered or placed. Adhered sensors are held in place by adhesive measures or by belt or belt equivalents. Adhered sensors are designed to be wearable for extended periods of time usually restricted by the battery life or power consumption of the sensor. Wearable's power consumption can be maximized through several means including: low-power circuit component choices, efficient software, and duty-cycling (i.e minimizing the on-time and maximizing the off-time). Heart failure patient indicators and symptoms are slowly changing with respect to circuit electronic performance and therefore lend themselves to duty-cycling for conserving power. It can be expected that a typical usage pattern could be to measure signals for 30 to 120 seconds two to four times per day rather than continuous measurement. If power consumption is optimized, the measurement life-cycle will be limited by the patient's tolerance for wearing an adhesive on the skin, which is usually no more than 7 to 14 days.

After any processing of the signal in the heart monitoring device, for example to remove ambient noise, the recorded signal is transmitted to the cloud for data processing and data archiving. To do so, several technologies are available including, as examples, cellular radio, Wi-Fi through a computer network, or BlueTooth™ to a smartphone or other device.

In some embodiments, several forms of signal processing can be necessary to provide information useful to the physician's diagnosis. The first processing can likely remove ambient noise by one of two methods. First, ambient noise can be recorded by a separate microphone and its signal subtracted from the cardiac and respiratory signals. The second method would be by active noise cancellation where the ambient signal is inverted and injected into the sensor's recording microphone. This method was made common by the Bose Corporation through their noise-cancelling headsets.

After noise reduction, the clinically standard heart sounds (S1, S2, S3 and S4) can be located by peak detection and/or frequency analysis. Peak detection can be enhanced if EKG signals are also captured by first finding R-waves in the EKG and verifying S1 and S2 detection as they happen relatively synchronously.

As shown in FIG. 7 (normal patient) and FIG. 8 (abnormal patient), there is a well-known acoustic indicator of heart failure in the form of the presence of an additional hear sound. This additional sound is referred to as the third heart sound (S3) or “ventricular gallop” (see FIG. 8). S3 occurs approximately 120 to 180 milliseconds after S2 when the mitral valve opens, allowing passive filling of the left ventricle. The large volume of blood striking a very compliant left ventricle, common in HF patients, causes the S3 sound.

The S3 sound is also often found in normal people less than 40 years of age and some trained athletes but should disappear before middle age and the onset of HF.

In some embodiments, it is possible that an S4 sound occurs in HF patients and the sound is low enough in frequency, i.e. between 0 and 20 Hz, to be inaudible by the human ear. This frequency range is also below the range of typical microphones used in stethoscopes and is best acquired with low-frequency accelerometers. S4 is a low-frequency sound that occurs coincident with late diastolic filling of the ventricle due to atrial contraction. Late in the filling cycle means that S4 occurs shortly before S1. S4 is caused by thickened left ventricular wall usually associated with hypertension or aortic stenosis, the former likely in HF patients and therefore its abrupt presence (i.e. being new in a sensor recording relative to previous recordings) can indicate a deteriorating condition for the patient.

S4 sound analysis is one of many factors, like changes in S1-S3, that the system can use to indicate the onset of HF complications perhaps before the patient becomes symptomatic. With the system storing previous recordings in a database, a baseline condition can be established and easily updated. With the patient serving as their own control, analysis of the acoustic signal can emphasize relative changes from baseline indicating improving or deteriorating conditions.

Again with the patient serving as their own control, the system may also act as an indicator of medication compliance or a means to evaluate the performance of personalized medicines.

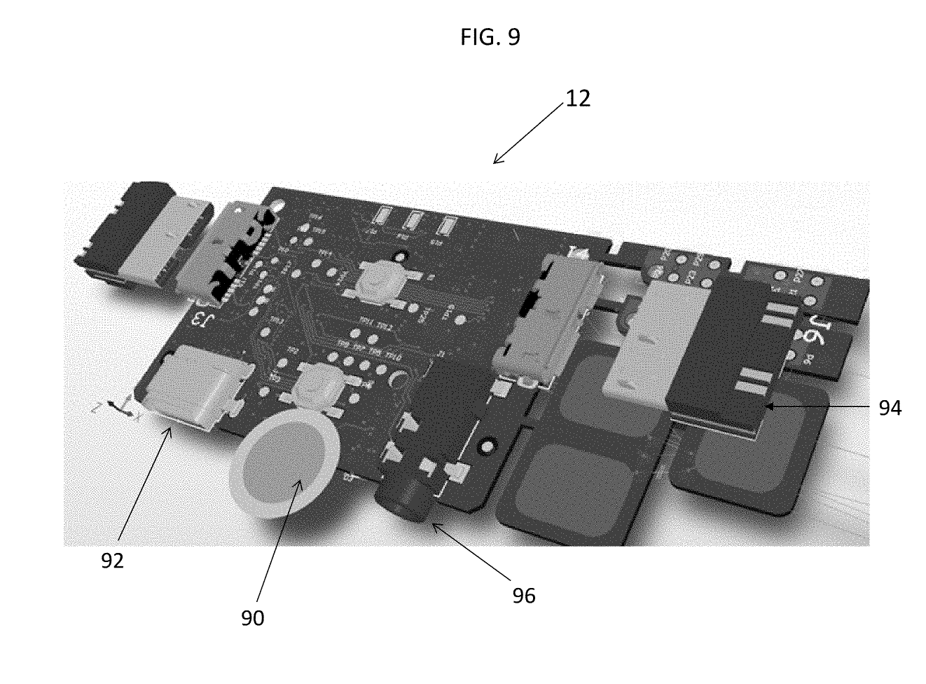

In reference to FIG. 9, the acoustic sensor includes one or more external microphones 90 (microphone or accelerometer), a charger interface 92, data storage 94 and audio output jack 96.

In some embodiments, the system can also be used in conjunction with other biologic measurements such as ejection fraction (EF) or cardiac blood pressure measurements that are routinely required of HF patients. Also, patient metadata such as age and weight can also be collected and analyzed with the sensor data in large patient populations. Additional inputs including physician evaluation of patient condition and tracking patients that do need medical intervention can set up favorable conditions for machine learning. With machine learning, the patient need not serve as their own control but the large populations with similar symptoms or inputs can be mathematically analyzed for statistical predictors of impending re-hospitalization. This predictive output suggesting the need for intervention is the novel benefit of such a system.

The system closes the loop to the physician for diagnosis and potential intervention through access to the cloud. The physician may access the data through computer or mobile technology software applications. With the loop to the healthcare provider closed, appropriate intervention can occur potentially saving the cost of hospital or emergency room admissions, which today represent the largest medical expense in the US healthcare system.

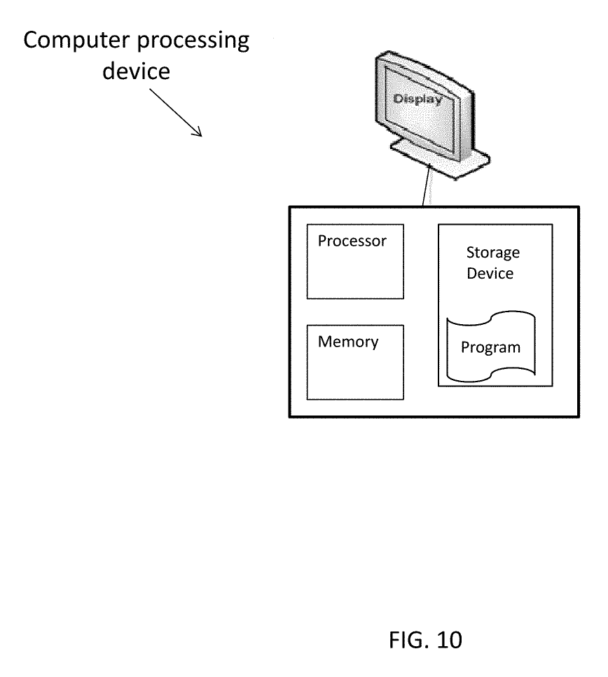

FIG. 10 shows, by way of example, a diagram of a typical processing architecture, which may be used in connection with the methods and systems of the present disclosure. A computer processing device can be coupled to a display for graphical output. The processing device includes a processor or microprocessor capable of executing software. Typical examples can be computer processors (such as Intel® or AMD® processors), ASICs, microprocessors, and the like. The processor can be coupled to a memory, which can be typically a volatile RAM memory for storing instructions and data while the processor executes. The computer processor may also be coupled to a storage device, which can be a non-volatile storage medium, such as a hard drive, FLASH drive, tape drive, DVDROM, or similar device. Although not shown, the computer processing device typically includes various forms of input and output. The I/O may include network adapters, USB adapters, Bluetooth radios, mice, keyboards, touchpads, displays, touch screens, LEDs, vibration devices, speakers, microphones, sensors, or any other input or output device for use with a computer processing device. The computer processor may also be coupled to other type of computer-readable media, including, but are not limited to, an electronic, optical, magnetic, or other storage or transmission device capable of providing a processor, such as the processor, with computer-readable instructions. Various other forms of computer-readable media can transmit or carry instructions to a computer, including a router, private or public network, or other transmission device or channel, both wired and wireless. The instructions may comprise code from any computer-programming language, including, for example, C, C++, C#, Visual Basic, Java, Python, Perl, and JavaScript.

The program can be a computer program or computer readable code containing instructions and/or data, and can be stored on a storage device. The instructions may comprise code from any computer-programming language, including, for example, C, C++, C#, Visual Basic, Java, Python, Perl, and JavaScript. In a typical scenario, the processor may load some or all of the instructions and/or data of the program into the memory for execution. The program can be any computer program or process including, but not limited to a web browser, a browser application, an address registration process, an application, or any other computer application or process. The program may include various instructions and subroutines, which, when loaded into the memory and executed by the processor, cause the processor to perform various operations, some or all of which may effectuate the methods for managing medical care disclosed herein. The program may be stored on any type of non-transitory computer readable medium, such as, without limitation, hard drive, removable drive, CD, DVD or any other type of computer-readable media.

Certain aspects of the present disclosure include process steps and instructions described herein in the form of an algorithm. It should be noted that the process steps and instructions of the present disclosure could be embodied in software, firmware or hardware, and when embodied in software, could be downloaded to reside on and be operated from different platforms used by a variety of operating systems. The disclosure can also be in a computer program product which can be executed on a computing system.

The present disclosure also relates to an apparatus for performing the operations herein. This apparatus may be specially constructed for the required purposes, or it may comprise a general-purpose computer selectively activated or reconfigured by a computer program stored in the computer. Such a computer program may be stored in a tangible computer-readable (or machine-readable) storage medium, such as, but not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each coupled to a computer system bus. Furthermore, the computers referred to in the specification may include a single processor or may be architectures employing multiple processor designs for increased computing capability. In some embodiments, the computer is connected to a display to display the images generated by the instant methods.

The algorithms and displays presented herein are not inherently related to any particular computer or other apparatus. Various general-purpose systems may also be used with programs in accordance with the teachings herein, or it may prove convenient to construct more specialized apparatus to perform the required method steps. The required structure for a variety of these systems will appear from the description. In addition, the present disclosure is not described with reference to any particular programming language. It will be appreciated that a variety of programming languages may be used to implement the teachings of the present disclosure as described herein, and any references to specific languages are provided for disclosure of enablement and best mode of the present disclosure.

As will be understood by those familiar with the art, the invention may be embodied in other specific forms without departing from the spirit or essential characteristics thereof. Likewise, the particular naming and division of the modules, features, attributes, methodologies, managers and other aspects are not mandatory or significant, and the mechanisms that implement the invention or its features may have different names, divisions and/or formats. Furthermore, as will be apparent to one of ordinary skill in the relevant art, the modules, features, attributes, methodologies, managers and other aspects of the invention can be implemented as software, hardware, firmware or any combination of the three. Of course, wherever a component of the present invention is implemented as software, the component can be implemented as a standalone program, as part of a larger program, as a plurality of separate programs, as a statically or dynamically linked library, as a kernel loadable module, as a device driver, and/or in every and any other way known now or in the future to those of skill in the art of computer programming. Additionally, the present invention is in no way limited to implementation in any specific programming language, or for any specific operating system or environment.

All patents, patent applications, and published references cited herein are hereby incorporated by reference in their entirety. It should be emphasized that the above-described embodiments of the present disclosure are merely possible examples of implementations, merely set forth for a clear understanding of the principles of the disclosure. Many variations and modifications may be made to the above-described embodiment(s) without departing substantially from the spirit and principles of the disclosure. It can be appreciated that several of the above-disclosed and other features and functions, or alternatives thereof, may be desirably combined into many other different systems or applications. All such modifications and variations are intended to be included herein within the scope of this disclosure.

What is claimed is:

1. A wearable heart monitoring device comprising:
one or more acoustic sensors, including
at least one microphone configured to operate in a frequency range related to human hearing; and
at least one accelerometer configured to operate in the range of low or sub-audible frequencies.

2. The wearable heart monitoring device of claim 1, further comprising a processor in communication with the one or more acoustic sensors, the processor being configured to analyze data collected by the one or more sensors to determine a condition of a heart.

3. The wearable heart monitoring device of claim 2, wherein analyzing the collected data includes noise reduction and location of clinically standard heart sounds using peak detection and frequency analysis.

4. The wearable heart monitoring device of claim 2, wherein the processor is positioned within the wearable heart monitoring device.

5. The wearable heart monitoring device of claim 2, wherein the processor is remote form the heart monitoring device and is configured to communicate with the one or more acoustic sensors using a wireless connection.

6. The wearable heart monitoring device of claim 2, wherein the processor is configured to sample data from the one or more acoustic sensors at a rate of 12 to 24-bits per sample.

7. The wearable heart monitoring device of claim 1, further comprising one or more data acquisition components configured to supplement the acoustic data collected by the one or more acoustic sensors with additional data relating to the heart.

8. The wearable heart monitoring device of claim 6, wherein one of the one or more data acquisition components is one or more EKG electrodes.

9. The wearable heart monitoring device of claim 7, wherein the one or more EKG electrodes are configured to collect EKG vectors to produce at least one EKG signal that can be analyzed for heart rate, R-wave detection, heart-rate variability, or the presence of arrhythmia.

10. The wearable heart monitoring device of claim 6, wherein one of the one or more data acquisition components is at least one gyroscope configured to determine an orientation of the heart monitoring device in space.

11. The wearable heart monitoring device of claim 8, wherein the gyroscope is configured to account for changes in blood flow through the heart relating to gravity as a body moves through space.

12. The wearable heart monitoring device of claim 8, wherein the gyroscope utilizes as least three axes to determine orientation of the heart monitoring device.

13. The wearable heart monitoring device of claim 1, further comprising a sensor recharger configured to refresh the acoustic sensor using wired or wireless communication.

14. The wearable heart monitoring device of claim 1, wherein the at least one microphone is configured to operate in the range between about 4 KHz and about 1 KHz.

15. The wearable heart monitoring device of claim 14, wherein the at least on accelerometer is configured to operate in the range between about 1 KHz and about 0.5 Hz.

16. A wearable heart monitoring device comprising:
one or more acoustic sensors, including
at least one microphone configured to operate in a frequency range related to human hearing; and
at least one accelerometer configured to operate in the range of low or sub-audible frequencies; and
at least one gyroscope configured to determine an orientation of the heart monitoring device in space,
wherein data collected by the one or more acoustic sensors and the at least one gyroscope is analyzed to determine a condition of a heart of a patient wearing the heart monitoring device.

17. The wearable heart monitoring device of claim 16, further comprising one or more data acquisition components configured to supplement the acoustic data collected by the one or more acoustic sensors with additional data relating to the heart.

18. The wearable heart monitoring device of claim 16, wherein one of the one or more data acquisition components is one or more EKG electrodes.

19. A method for monitoring heart activity, comprising:
collecting acoustic data using one or more acoustic sensors, the one or more acoustic sensors including at least one microphone configured to operate in a frequency range related to human hearing and at least one accelerometer configured to operate in the range of low or sub-audible frequencies; and
analyzing data collected by the one or more sensors to determine a condition of a heart using a processor.

20. The method of claim 19, wherein analyzing the collected data includes noise reduction and location of clinically standard heart sounds using peak detection and frequency analysis.
